(19) United States
(12) Patent Application Publication
(10) Pub. No.: US 2024/0123019 A1
(43) Pub. Date: Apr. 18, 2024

(54) COMPOSITION FOR TREATING OR AMELIORATING LIVER DISEASE AND LIVER DYSFUNCTION COMPRISING ZIZANIA LATIFOLIA EXTRACT

(71) Applicant: BTC CORPORATION, Ansan-si (KR)

(72) Inventors: Tae Young KIM, Ansan-si (KR); Joo Myung MOON, Ansan-si (KR)

(73) Assignee: BTC CORPORATION, Ansan-si (KR)

(21) Appl. No.: 18/396,645

(22) Filed: Dec. 26, 2023

Related U.S. Application Data
(63) Continuation of application No. PCT/KR2022/006967, filed on May 16, 2022.

(30) Foreign Application Priority Data
Jul. 12, 2021 (KR) 10-2021-0091107

(51) Int. Cl. A61K 36/899; A23L 33/105; A61P 1/16

(52) U.S. Cl. CPC A61K 36/899; A23L 33/105; A61P 1/16

(57) ABSTRACT

The present invention relates to a composition for treating or ameliorating a liver disease and liver dysfunction, which includes a Zizania latifolia extract and a pharmaceutical composition or a food composition. The Zizania latifolia extract according to the present invention contains a high content of tricin, and thus may have an improved treatment, alleviation, amelioration, or prevention effect on liver diseases and liver dysfunction compared to those of conventional extraction methods.

### CROSS-REFERENCE TO RELATED APPLICATION

This application is based on the PCT Application No. PCT/KR2022/006967, filed on May 16, 2022, and claims the benefit of priority from the prior Korean Patent Application No. 10-2021-0091107, filed on Jul. 12, 2021, the disclosures of which is incorporated herein by reference in its entirety.

### BACKGROUND

### 1. Field of the Invention

The present invention relates to a composition for treating or ameliorating a liver disease and liver dysfunction, which includes a Zizania latifolia extract and a pharmaceutical composition or food composition. The Zizania latifolia extract according to the present invention contains a high content of tricin, and thus may have an improved treatment, alleviation, amelioration, or prevention effect on liver diseases and liver dysfunction.

### 2. Discussion of Related Art

The liver is an important organ that performs blood storage and circulation, metabolism of lipids and the like, excretion of bile, storage of various nutrients, regulation of blood flow, and detoxification in the human body. However, the liver becomes overloaded as our body is exposed to various pollutants and toxic substances, and serious damage to the liver is also caused by mental stress, excessive drinking, smoking, and the like.

Because the liver is the first defense organ that prevents damage from ingestion of foreign substances, a decrease in liver function may cause severe diseases due to viruses or various drugs. This may cause abnormalities in the immune system, thereby causing other diseases. In particular, acute or chronic disorders occur due to various causes such as excessive consumption of food containing fatty ingredients or alcohol, viral infections, harmful substances such as various drugs, nutritional deficiencies, and the like, which may cause fatty liver, hepatitis, jaundice, cirrhosis, liver cancer, and the like. Also, excessive fat intake through food or excessive alcohol intake causes fatty liver in which lipids accumulate in the liver tissue. In this case, aspartate aminotransferase (AST or GOT), alanine aminotransferase (ALT or GPT), and γ-glutamyltransferase (γ-GPT), and the like increase in the serum.

Liver diseases are classified according to the cause of the disease into viral liver diseases, alcoholic liver diseases, toxic liver diseases, fatty liver diseases, autoimmune liver diseases, metabolic liver diseases, and other liver diseases. Because the liver is an organ with a large buffering capacity, there are many cases of disease that are not recognized in the early stages. Also, because the liver disease is not found until the disease has progressed too far, it is the leading cause of death not only in Korea but also around the world.

As described above, the loss of liver function is unconscious and causes many problems in the human body by causing the loss of the body's defense and detoxification functions. Therefore, there is a need for development of medicines and health functional foods having hepatoprotective effects, which are manufactured into safe preparations without side effects.

Meanwhile, Zizania latifolia Turcz. is a perennial grass belonging to the Gramineae family and is called Zizania. It is known to be effective against diseases such as hypertension, stroke, constipation, obesity, arteriosclerosis, and the like. Also, in folk medicines, it is known that drinking a decoction of Zizania latifolia Turcz. or drinking the juice thereof is effective for pesticide poisoning, chemical poisoning, food poisoning, and the like. Also, healthy foods and liquid tea using Zizania latifolia Turcz. have been developed recently (Korean Patent Publication No. 10-2010-0104334, Korean Patent Publication No. 10-2009-0127970).

Because the active substances contained in Zizania latifolia Turcz. are glycosides of tricin and derivatives thereof that are bound to highly hydrophilic sugar molecules, the active substances may have a low absorption rate in the body due to their low intestinal cell membrane lipid permeability. However, because the carbon skeleton itself, except for the sugar molecules, has high hydrophobicity, it has high solubility in the intestinal membrane lipids, which makes it easy to permeate the intestinal cell membrane. Therefore, when the glycosides of tricin and derivatives thereof, which are likely to lower the intestinal absorption rate, are converted into tricin, they may be expected to have high activity.

Also, there are documents disclosing that the desired activity is further enhanced when various flavonoid glycosides are converted into aglycones. In this regard, it is known that when the glycosides of tricin and derivatives are converted into aglycones, the tricin and derivatives also have further enhanced whitening, wrinkle improvement, anti-inflammatory, anti-allergy, and moisturizing effects.

However, only a small portion of the pharmacological effects of the Zizania latifolia extract have been found so far, and studies on the hepatoprotective effects of Zizania latifolia Turcz. have also been hardly explored. In addition, the pharmacological activity or optimal pharmacological effects based on their main components and contents thereof are not yet known. Korea Patent Publication No. 10-2016-0076145 discloses a health food for relieving hangovers, which includes a Zizania latifolia extract and a Zizania latifolia-derived culture solution of effective microorganisms. However, there are limitations in that there is no research on the mechanism of action at the molecular biology level, and there is no explanation of the effective microorganisms. Moreover, in the case of fermentation, microorganisms react differently to a slight difference in composition, which makes it very difficult to control their metabolites. Accordingly, there is a need to develop an enzyme treatment method that may allow an enzyme to specifically react with the active ingredients (i.e., tricin and tricin glycosides) to consistently obtain metabolites. A large number of natural products, including Zizania latifolia Turcz., may have a significant difference in effectiveness depending on the differences in components or contents contained in the natural products. In order to obtain the desired effects, there is a need for research on the natural products themselves, as well as their compositions, contents, and the like that may achieve the optimal effects.

### SUMMARY OF THE INVENTION

Under the above technical background, the present applicant has confirmed that the active ingredients contained in a Zizania latifolia extract and an enzyme-treated extract of Zizania latifolia Turcz. exhibit a pharmacological effect such as treating or improving a liver disease and liver dysfunction. Therefore, the present invention has been completed based on these facts.

Accordingly, an object of the present invention is to provide a pharmaceutical composition for treating or ameliorating a liver disease and liver dysfunction, which includes a Zizania latifolia extract.

Another object of the present invention is to provide a food composition for ameliorating or preventing a liver disease and liver dysfunction, which includes a Zizania latifolia extract.

According to one embodiment, the Zizania latifolia extract may include an enzyme-treated extract of Zizania latifolia Turcz., and a polar or non-polar solvent extract of the residue remaining after obtaining the enzyme-treated extract of Zizania latifolia Turcz.

Here, the enzyme-treated extract of Zizania latifolia Turcz. may be an extract obtained by treating Zizania latifolia Turcz. with at least one enzyme selected from a pectinase, a hemicellulase, an arabinanase, an arabanase, a cellulase, a beta-glucanase, and a xylanase.

The Zizania latifolia extract may include at least one selected from a flavonoid glycoside and a flavonoid aglycone. More particularly, the Zizania latifolia extract may include at least one selected from tricin, tricin-7-O-β-D-glucopyranose, salcolin A, sacolin B, sacolin C, salcolin D, tricin-4′-O-(threo-β-guaiacylglyceryl) ether 7-O-β-D-glucopyranose, tricin-4′-O-(erythro-β-guaiacylglyceryl) ether 7-O-β-D-glucopyranose, tricin-4′-O-(threo-β-guaiacylglyceryl) ether 7″-O-β-D-glucopyranose, and tricin-4′-O-(erythro-β-guaiacylglyceryl) ether 7″-O-β-D-glucopyranose.

In order to address the above technical problems, according to one aspect of the present invention, there is provided a pharmaceutical composition for treating or ameliorating a liver disease and liver dysfunction, which includes a Zizania latifolia extract.

Here, the liver disease may be at least one selected from fatty liver, liver fibrosis, liver cirrhosis, liver cancer, jaundice, and an inflammatory liver disease.

In this case, the inflammatory liver disease may be at least one selected from hepatitis, acute hepatitis, chronic hepatitis, alcoholic hepatitis, non-alcoholic hepatitis, subacute hepatitis, viral hepatitis, toxic liver disease, liver abscesses, granulomatous hepatitis, autoimmune hepatitis, and lupoid hepatitis, and the liver dysfunction may be caused by at least one selected from hangovers, alcohol-induced liver damage, toxic substance-induced liver damage, and hepatocyte damage.

According to another aspect of the present invention, there is provided a food composition for ameliorating or preventing a liver disease and liver dysfunction, which includes a Zizania latifolia extract.

A Zizania latifolia extract according to the present invention is very effective in treating or ameliorating liver diseases and liver dysfunction, and thus can be expected to prevent various liver diseases and liver dysfunction such as fatty liver, liver fibrosis, liver cirrhosis, liver cancer, jaundice, and inflammatory liver diseases, or have a liver function improvement effect of restoring liver function to normal levels.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows the test results of cell survival rates in HepG2 cells in which hepatotoxicity is induced in Experimental Example 1.

FIG. 2 shows the test results of the ability to inhibit ROS production in HepG2 cells in which hepatotoxicity is induced in Experimental Example 1.

FIG. 3 shows the results of measuring the expression levels of alcohol dehydrogenase (ADH) and acetaldehyde dehydrogenase (ALDH) in HepG2 cells in Experimental Example 1.

FIG. 4 is a graph showing the results of measuring the blood concentrations of ALT, AST, and ALP, which are liver function-related enzymes indicating liver cell damage, in Experimental Example 2.

FIG. 5 shows the Oil Red O & Hematoxylin results for confirming a lipid level in the liver in Experimental Example 2.

FIG. 6 shows the TG (triglyceride) and total cholesterol concentrations in liver tissue and serum in Experimental Example 2.

FIG. 7 shows the results of measuring the degrees of activation of lipid peroxide and non-enzymatic antioxidant systems in the liver tissue in Experimental Example 2.

FIG. 8 shows the results of measuring ADH and ALDH activities in the liver tissue after administration of alcohol in Experimental Example 2.

FIG. 9 shows the results of measuring changes in blood concentrations of alcohol and acetaldehyde in Experimental Example 2.

FIG. 10 shows the results of measuring the Cₘₐₓ values of alcohol and acetaldehyde in Experimental Example 2.

FIG. 11 shows the results of measuring the ability to scavenge DPPH and superoxide radicals in Experimental Example 3.

### DETAILED DESCRIPTION OF EXEMPLARY EMBODIMENTS

In order to better understand the present invention, certain terms are defined herein for the sake of convenience. Unless defined otherwise herein, scientific and technical terms used herein will have meanings commonly understood by those of ordinary skill in the art. In addition, unless specifically indicated otherwise in the context, it should be understood that terms in their singular form also include plural forms, and terms in their plural form include singular forms as well.

Hereinafter, compositions for treating or ameliorating a liver disease and liver dysfunction according to some embodiments of the present invention will be described in further detail.

According to one aspect of the present invention, a pharmaceutical composition for treating or ameliorating a liver disease and liver dysfunction, which includes a Zizania latifolia extract, may be provided.

According to one embodiment of the present invention, the liver disease may be one or more selected from the group consisting of fatty liver, liver fibrosis, liver cirrhosis, liver cancer, jaundice, and an inflammatory liver disease, and the inflammatory liver disease may be one or more selected from the group consisting of hepatitis, acute hepatitis, chronic hepatitis, alcoholic hepatitis, non-alcoholic hepatitis, subacute hepatitis, viral hepatitis, toxic liver disease, liver abscesses, granulomatous hepatitis, autoimmune hepatitis, and lupoid hepatitis, but the present invention is not limited thereto.

According to one embodiment of the present invention, the liver dysfunction may be caused by one or more selected from the group consisting of hangovers, alcohol-induced liver damage, toxic substance-induced liver damage, and hepatocyte damage, but the present invention is not limited thereto.

As used throughout this specification, the term “treatment” refers to any action that improves or benefits the symptoms of a liver disease or liver dysfunction by administering a pharmaceutical composition including the Zizania latifolia extract of the present invention as an active ingredient to a subject suffering from the liver disease or liver dysfunction.

According to one embodiment of the present invention, the Zizania latifolia extract may treat, ameliorate or prevent a liver disease or liver dysfunction by improving liver function.

Here, the liver function improvement refers to the recovery and treatment of weakened liver function, and, for example, includes amelioration of liver dysfunction caused by hangovers, liver cell damage, alcoholic liver, hepatotoxic substance-induced liver damage or detoxification, and the like, as well as the prevention and treatment of liver damage.

Also, it includes improving all or part of the liver's various functions such as continuous bile secretion function, synthesis function, excretion function, and metabolic function due to lifestyle habits such as drinking, a high-fat diet, overwork, and the like. Especially, it includes restoring, as the liver function improvement, levels of the biomarkers AST and ALT, which indicate liver parenchymal cell damage, γ-GPT and ALP, which indicate damage to the biliary tract, and bilirubin, which indicates damage to the bile secretion function, to normal levels.

As an example of the liver function improvement, the improvement or curing of hangovers suppresses or reduces headaches, diarrhea, loss of appetite, nausea, vomiting, chills, cold sweat, and the like that appear after drinking alcohol, restores the body from decreased cognitive ability and decreased exercise ability, and maintains the normal hematological and hormonal status.

For example, groups in need of liver function improvement include ordinary people in need of liver disease prevention and liver health promotion; patients in need of liver disease prevention and liver health promotion who have a prognosis for liver disease or whose liver disease is not recognized in the early stages of the disease; or patients in need of liver disease treatment or liver function improvement to enhance an action of liver detoxification, who suffer from diseases such as fatty liver, hepatitis, jaundice, liver cirrhosis, liver cancer, and the like. In this case, when the patients are in need of liver function improvement, it may be used without any limitation.

The Zizania latifolia extract used in the present invention is a safe natural preparation made from Zizania latifolia Turcz., and unlike general single compounds, it is possible to improve indicators associated with liver health without side effects when applied to the human body.

According to one embodiment of the present invention, the Zizania latifolia extract has excellent antioxidant ability, and thus may effectively suppress oxidative stress (reactive oxygen species and the like) in the liver.

According to one embodiment of the present invention, the Zizania latifolia extract has an excellent ability to protect liver cells against toxic substances, may prevent liver and liver cell damage and may treat/improve a damaged liver or liver cells.

According to one embodiment of the present invention, the Zizania latifolia extract has an excellent ability to metabolize alcohol, and thus may effectively lower the concentration of alcohol in the blood and liver tissue.

According to one embodiment of the present invention, the Zizania latifolia extract may effectively suppress the production of triglycerides in liver tissue, and thus may effectively treat, prevent, or improve alcoholic or non-alcoholic fatty liver or steatohepatitis that may be caused therefrom.

According to one embodiment of the present invention, the Zizania latifolia extract may include an enzyme-treated extract of Zizania latifolia Turcz.; and a polar or non-polar solvent extract of the residue remaining after obtaining the enzyme-treated extract of Zizania latifolia Turcz.

The enzyme used for the enzymatic treatment may include one or more selected from the group consisting of a pectinase, a hemicellulase, an arabinanase, an arabanase, a cellulase, a beta-glucanase, and a xylanase.

As the Zizania latifolia extract used herein is manufactured using a method described below, the sugars of tricin glycosides and derivative glycosides in the Zizania latifolia extract may be decomposed and converted into aglycones having enhanced bioavailability and exhibiting superior activity to the glycosides of tricin and derivatives thereof. As a result, the absorption rate in the body of the ingredients effective in improving liver function in the Zizania latifolia extract may be improved.

According to one embodiment of the present invention, the Zizania latifolia extract may include at least one selected from a flavonoid glycoside and a flavonoid aglycone.

Specifically, the Zizania latifolia extract may include at least one selected from tricin, tricin-7-O-β-D-glucopyranose, salcolin A, sacolin B, sacolin C, salcolin D, tricin-4′-O-(threo-β-guaiacylglyceryl) ether 7-O-β-D-glucopyranose, tricin-4′-O-(erythro-β-guaiacylglyceryl) ether 7-O-β-D-glucopyranose, tricin-4′-O-(threo-β-guaiacylglyceryl) ether 7″-O-β-D-glucopyranose, and tricin-4′-O-(erythro-β-guaiacylglyceryl) ether 7″-O-β-D-glucopyranose. Also, the content of tricin in the Zizania latifolia extract may be in a range of 0.1% by weight to 90% by weight.

Meanwhile, according to one embodiment of the present invention, the pharmaceutical composition may optionally include at least one selected from vitamin B, vitamin C, vitamin E, beta-carotene, Ca, Mg, Zn, lecithin, alanine, taurine, maltol, fructose, an oligosaccharide, Ganoderma lucidum, glutamate, chitosan, aspartic acid, Cordyceps militaris, a Hovenia dulcis extract, an Alnus borealis extract, and a milk thistle extract in addition to the Zizania latifolia extract.

Also, the pharmaceutical composition may further include a pharmaceutically acceptable carrier in addition to the Zizania latifolia extract. The pharmaceutical composition including the Zizania latifolia extract and the carrier may be prepared into oral or parenteral formulations according to conventional preparation methods.

Here, the term “pharmaceutically acceptable” means that it has no toxicity beyond what is acceptable for a subject to be applied (prescribed) without inhibiting the activity of the active ingredient.

According to one embodiment of the present invention, the pharmaceutical composition may include a pharmaceutically effective amount of the Zizania latifolia extract.

Here, the term “pharmaceutically effective amount” refers to an amount sufficient to treat or prevent a disease at a reasonable benefit/risk ratio applicable to medical treatment or prevention, and the effective dose level may be determined depending on factors including the severity of a disease, the activity of a drug, the age, body weight, health, and gender of a patient, the sensitivity of a patient to the drug, the time of administration of the composition of the present invention used, the route of administration and the rate of excretion, the duration of treatment, drugs mixed or used concurrently with the composition of the present invention used herein, and other factors well-known in the medical field.

The Zizania latifolia extract used herein may be prepared by (1) adding water to Zizania latifolia Turcz. and immersing the Zizania latifolia Turcz. in hot water; (2) treating the immersion product, which is cooled, after the hot-water immersion in step (1) with one or more enzymes selected from the group consisting of a pectinase, a hemicellulase, an arabinanase, an arabanase, a cellulase, a beta-glucanase, and a xylanase to allow the immersion product to react with the enzymes and then filtering the reaction mixture to obtain an enzyme-treated extract; (3) extracting the residue remaining after the filtration in step (2) with any one solvent selected from the group consisting of water, a C₁ to C₄ lower alcohol, and a mixture thereof to obtain a secondary extract; and (4) mixing the enzyme-treated extract of step (2) with the secondary extract of step (3) and then concentrating or drying the resulting mixture.

In step (1), the hot-water immersion may be carried out at 20° C. to 130° C. for 5 minutes to 100 hours after adding 1 to 100 parts by weight of water to 1 part by weight of Zizania latifolia Turcz., more preferably carried out at 50 to 120° C. for 30 minutes to 100 hours after adding 1 to 50 parts by weight of water to 1 part by weight of Zizania latifolia Turcz., and most preferably carried out at 70° C. to 120° C. for 30 minutes to 4 hours after adding 5 to 30 parts by weight of water to 1 part by weight of Zizania latifolia Turcz.

In step (2), cooling may be performed at 10° C. to 90° C., preferably 20° C. to 45° C.

Also, in step (2), 100 parts by weight of the immersion product may be treated with 0.01 to 80 parts by weight of enzyme. In this case, when the content of enzyme is below the above numerical range, an amount of enzyme is too small, which makes it difficult to smoothly separate tricin present in the hot-water immersion product of Zizania latifolia Turcz. from a tricin precursor and convert the tricin precursor into tricin. On the contrary, when Zizania latifolia Turcz. is treated with an excessive amount of enzyme, substances that are not involved in activity, other than the tricin precursor, are extracted together at high contents. In this case, an extract having a relatively low content of tricin and reduced efficacy may be prepared, and may affect the cost of the manufacturing process, which is not commercially desirable.

In this case, in step (2), the immersion product may be treated with the enzyme and reacted at 10° C. to 90° C. for 5 minutes to 120 hours, preferably treated with the enzyme and reacted at 20° C. to 80° C. for 5 minutes to 120 hours, more preferably treated with the enzyme and reacted at 30° C. to 60° C. for 5 minutes to 48 hours, and most preferably treated with the enzyme and reacted at 20° C. to 45° C. for 30 minutes to 24 hours. As a result, because the reaction time of the appropriate enzyme is inversely proportional to the amount of enzyme added, the greater the amount of enzyme added, the shorter the reaction time.

Meanwhile, the enzyme treatment method of step (2) may be performed by adding an enzyme directly to the extract to allow the extract to react with the enzyme, and inactivating the enzyme after the reaction, or may be performed as a continuous process using an enzyme immobilization method.

However, when the reaction time after the enzyme treatment is less than the above numerical range, the enzyme treatment time is too short, thereby reducing the conversion rate from the tricin precursor present in the hot-water immersion product of Zizania latifolia Turcz. into tricin. On the contrary, the enzyme treatment time is greater than the above numerical range, it may affect the cost of the manufacturing process, which is not commercially desirable.

In order to achieve the objects of the present invention, the complex of the enzymes may be used as a complex of purified single enzymes or as a combination of one or more commercialized complex enzymes. Examples of the commercialized enzymes may be Pectinex XXL and/or Viscozyme L.

According to one embodiment of the present invention, in step (2), the immersion product may be treated concurrently with three enzymes, for example, a beta-glucosidase, a cellulase, and a hemicellulase. In particular, when the three enzymes are mixed and used, the content and yield of various tricins are highest, and its functionality is also significantly superior, which is desirable.

According to one embodiment of the present invention, in step (2), the enzyme may convert flavonoid glycosides included in Zizania latifolia Turcz. into flavonoid aglycones. The glycosides of tricin and derivatives thereof are glycosides in which the sugar bonds are all R-O-β-D-glucopyranose bonds and glucose is bonded to the structure of tricin and derivatives thereof through a beta bond.

In step (3), 1 part by weight of the residue may be extracted at 20° C. to 130° C. for 10 minutes to 100 hours using 1 to 100 parts by weight of water or an aqueous alcohol solution as a solvent, preferably extracted at 20° C. to 100° C. for 2 hours to 12 hours. Alternatively, 1 part by weight of the residue may be extracted at 50° C. to 100° C. for 30 minutes to 10 hours using 1 to 30 parts by weight of water or an aqueous alcohol solution as the solvent. In this case, when the extraction temperature is less than the above range, the extraction yield of the tricin and active ingredients involved in activity may be lowered. On the other hand, when the extraction temperature is greater than the above range, the active ingredients may be destroyed or may be extracted together with the high contents of substances not involved in activity. As a result, an extract having reduced efficacy may be extracted, which is not desirable.

According to one embodiment of the present invention, the alcohol may be 5 to 95% by weight of an aqueous ethanol solution, preferably 20 to 80% by weight of an aqueous ethanol solution, more preferably 30 to 70% by weight of an aqueous ethanol solution, and most preferably 50% by weight of an aqueous ethanol solution.

That is, most preferably, in step (3), the extraction may be performed at 80° C. for 6 hours using 50% ethanol as the solvent. As a result, when the residue is extracted under these extraction conditions, all of the content, yield, and functionality of tricin may be maximized, which is preferable.

Also, in step (4), the mixture may be concentrated to obtain a concentrate, or the concentrate may be dried to obtain an extract in a powder form.

According to one embodiment of the present invention, a pretreatment step of irradiating the extract with ultrasonic waves or microwaves may be further performed prior to the extraction in step (3). In this case, the extract may be irradiated with a combination of ultrasonic waves and microwaves. When the enzyme-treated extract of Zizania latifolia Turcz. is irradiated with ultrasonic waves or/and microwaves, it is possible to obtain an extract having an improved liver function improvement effect compared to the case where the extract is not irradiated with ultrasonic waves or/and microwaves, which is desirable.

The ultrasonic waves may be irradiated at 15 to 25 kHz and 500 to 800 watts for 2 to 30 minutes, and the microwaves may be irradiated at 2,000 to 3,000 MHz and 50 to 400 watts for 5 to 60 seconds. As a result, when the irradiation energy and time of the ultrasonic waves or microwaves are less than the above ranges, the effect of irradiation is minimal. On the other hand, when the irradiation energy and time are greater than the above ranges, the extraction rate of substances not involved in activity may become higher, which is not desirable.

Meanwhile, impurities may be removed from the mixture using a conventional filtration method or device. For example, an extract from which the impurities have been removed may be obtained by filtration using a centrifugation method or using a filter or micro-filter. In this case, the filter may be a 1 to 200 μm filter, and the micro-filter may be a 0.2 to 0.8 μm filter, but the present invention is not limited thereto.

According to another aspect of the present invention, a food composition for ameliorating or preventing a liver disease and liver dysfunction, which includes the Zizania latifolia extract, may be provided. The content that overlaps with the above-described pharmaceutical composition may be equally applied to the food composition.

As used throughout this specification, the term “improvement” refers to all actions in which liver disease and liver dysfunction are improved or beneficially changed by administration of the composition.

As used throughout this specification, the term “food” includes meat, sausages, bread, chocolate, candies, snacks, confectioneries, pizza, ramen, other noodles, gum, dairy products including ice cream, various soups, beverages, tea, drinks, alcoholic beverages, vitamin complexes, health functional foods, and health foods. In this case, the food includes all foods in the conventional sense.

As used throughout this specification, the term “health functional food” refers to a food manufactured and processed using raw materials or ingredients having functionality useful to the human body in accordance with Act No. 6727 on Health Functional Food, and the term “functionality” means that a food has functions that bring particularly beneficial effects on health, as well as supplying nutrients, that is, functions such as biological defense, disease prevention and recovery, body rhythm regulation, and the like. The health functional food composition according to the present invention is effective in improving various liver functions as described above.

The food composition may be prepared by a method commonly used in the art, and may be prepared by adding raw materials and components commonly added during preparation in the art. Additionally, the food formulation may also be prepared without limitation as long as it is a formulation recognized as a food. The food composition of the present invention may be manufactured in various types of formulations. Unlike general drugs, the composition is prepared using a food as a raw material, and thus has an advantage of having no side effects that may occur when taking a drug for a long period of time, and is highly portable. Therefore, the food of the present invention may be consumed as a supplement to enhance the effect of improving the intestinal environment.

The food composition may further include a physiologically acceptable carrier. In this case, the type of carrier is not particularly limited, and any carrier commonly used in the art may be used. The additive may be selected depending on the type of food, and used in an appropriate amount.

The Zizania latifolia extract of the present invention may be added as is or may be used with other foods or food ingredients, and may be used properly according to conventional methods. The mixing amount of the active ingredient may be properly determined depending on its purpose of use (prevention, health, or therapeutic treatment). In general, when a food or beverage is prepared, the food composition of the present invention may be added in an amount of 50 parts by weight or less, particularly 20 parts by weight or less, relative to the food or beverage. However, when the extract is consumed for a long period of time for the purpose of health and hygiene, it may be included at the content below the above range. Because there is no problem in terms of safety, the active ingredient may be used in an amount greater than the above range.

When the food composition can be shown to ameliorate a liver disease or intestinal dysfunction by improving liver function, it may include the Zizania latifolia extract of the present invention in various weight percentages. Specifically, the Zizania latifolia extract of the present invention may be included at 0.00001 to 100% by weight or 0.01 to 80% by weight, based on the total weight of the food composition, but the present invention is not limited thereto.

### MODE FOR INVENTION

Hereinafter, the present invention will be described in further detail with reference to examples. However, it should be understood that these examples are only provided to illustrate the present invention, and are not intended to limit the scope of the present invention.

### Preparation Example: Method of Preparing Zizania Latifolia Extract

### Preparation Example 1

100 g of commercially available Zizania latifolia Turcz. was ground and weighed, and immersed in hot water at 80° C. for 2 hours after purified water was added at an amount 20-fold the weight of the Zizania latifolia Turcz. Thereafter, Pectinex XXL and Viscozyme L were weighed and mixed with the hot-water immersion product so that each of the amounts of Pectinex XXL and Viscozyme L was 0.3% by weight based on the amount of the administered Zizania latifolia Turcz., the pH was then adjusted to 4.5, and the resulting mixture was reacted at 35° C. for 4 hours. Subsequently, the enzyme was inactivated at 110° C. by primary filtration to obtain an enzyme-treated extract of Zizania latifolia Turcz.

Here, Pectinex XXL is a multi-enzyme complex including a pectinase, a hemicellulase, and an arabinase, and Viscozyme L is a multi-enzyme complex including an arabinanase, a cellulase, a beta-glucanase, a hemicellulose, and a xylanase. To the residue remaining after obtaining the enzyme-treated extract of Zizania latifolia Turcz., 50% (w/w) fermented alcohol was added at an amount that is 10-fold the weight of the residue. Then, the residue was extracted at 80° C. for 2 hours, followed by secondary filtration to obtain an alcohol extract of Zizania latifolia Turcz.

Next, the enzyme-treated extract of Zizania latifolia Turcz. and the alcohol extract of Zizania latifolia Turcz. were mixed, and sterilized to prepare a Zizania latifolia extract.

### Experimental Example 1: Evaluation of In Vitro Hepatoprotective Effect of Zizania Latifolia Extract

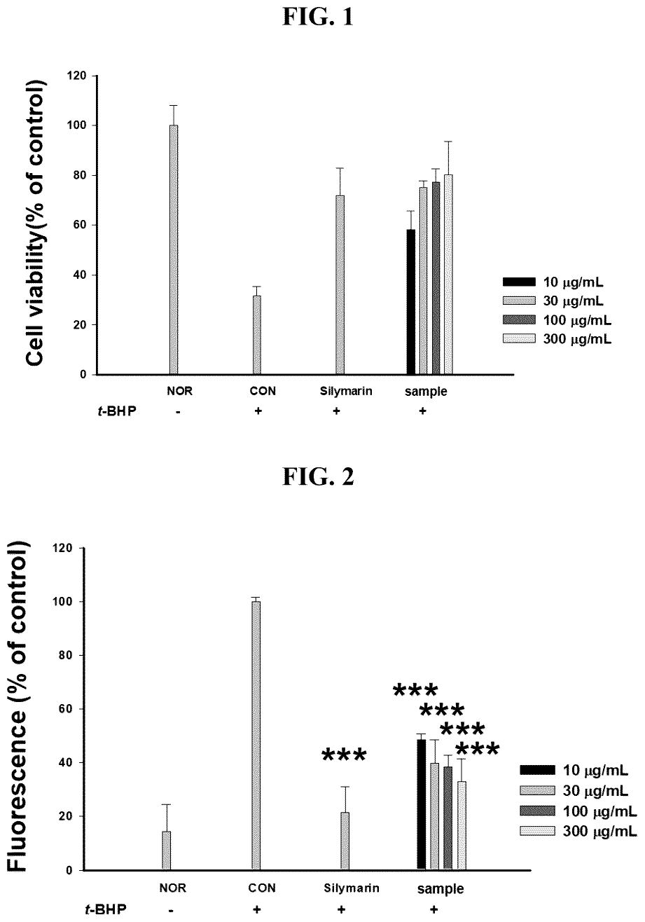

A control (CON) treated with t-BHP, which causes hepatotoxicity, in a liver cell line (e.g., HepG2 cells), a positive control (Silymarin) treated with silymarin as a positive control, and experimental groups (Sample) treated with different concentrations of the Zizania latifolia extract prepared according to Preparation Example 1 were tested for cell viability, and the results are shown in FIG. 1.

Referring to the results shown in FIG. 1, it was confirmed that the cell survival rate was higher at all concentrations in the experimental groups compared to the control, and the cell survival rate was higher at a concentration of 30 μg/mL or more in the experimental groups compared to the positive control.

Also, the same control, positive control, and experimental groups were tested for their ability to inhibit ROS production, and the results are shown in FIG. 2. Referring to the results shown in FIG. 2, it was confirmed that the ability to inhibit ROS production was observed at all concentrations in the experimental groups.

Based on the results of FIGS. 1 and 2, it can be seen that the Zizania latifolia extract exhibited a hepatoprotective effect in a concentration-dependent manner.

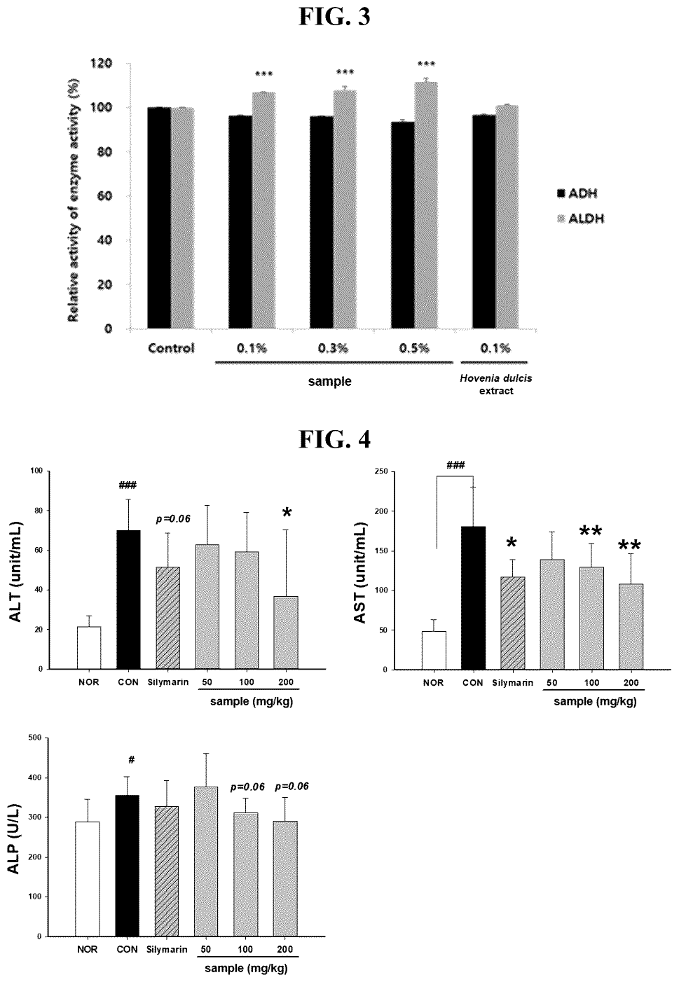

Meanwhile, a liver cell line (e.g., HepG2 cells) was treated with the Zizania latifolia extract prepared in Preparation Example 1 at concentrations of 0.1%, 0.3%, and 0.5% (experimental groups) to determine the expression levels of alcohol dehydrogenase (ADH) and acetaldehyde dehydrogenase (ALDH). A group treated with the Hovenia dulcis extract at a concentration of 0.1% was used as a positive control, and the results are shown in FIG. 3.

Referring to the results shown in FIG. 3, it can be seen that the expression level of ALDH was higher at all concentrations in the experimental groups compared to the positive control treated with the Hovenia dulcis extract. Based on these results, it can be expected that the Zizania latifolia extract of the present invention may sufficiently replace the Hovenia dulcis extract used as a liver functional material.

### Experimental Example 2: Evaluation of In Vivo Hepatoprotective Effect of Zizania Latifolia Extract

To determine the in vivo hepatoprotective effect, 6-week-old SD rats in which alcoholic fatty liver was induced were used. Specifically, ethanol was administered three times at 12-hour intervals to SD rats at a concentration of 5 g/kg in order to induce fatty liver.

Groups to which the Zizania latifolia extract prepared in Preparation Example 1 was administered at concentrations of 50, 100, and 200 mg/kg at 30 minutes before alcohol administration were used as experimental groups, a group to which 50 mg/kg of silymarin was administered in the same manner was used as a positive control, and a group to which alcohol was administered alone was used as a control.

Referring to FIG. 4, which shows the results of measuring blood concentrations of ALT, AST, and ALP, which are liver function-related enzymes indicating liver cell damage, it can be seen that the Zizania latifolia extract exhibited a hepatoprotective effect in a concentration-dependent manner. Also, it was confirmed that the blood concentrations of the liver function-related enzymes were lower at all concentrations in the experimental groups compared to the control, and the hepatoprotective effect was higher at a concentration of 200 mg/kg or more in the experimental groups compared to the positive control.

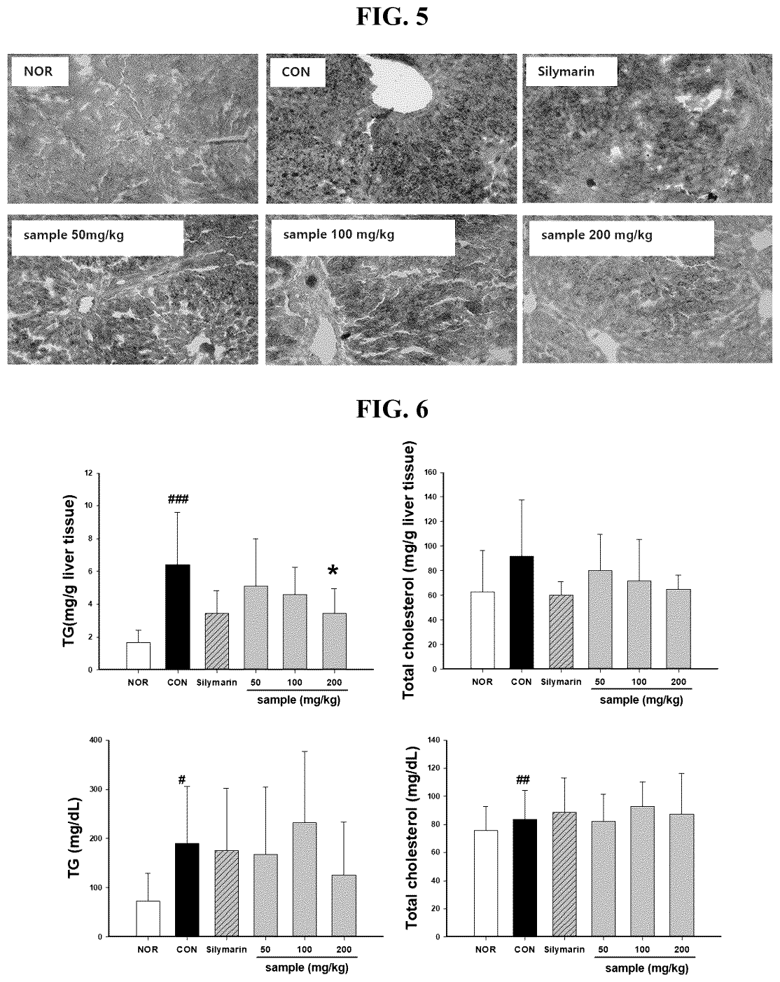

Also, referring to FIG. 5, which shows the Oil Red O & Hematoxylin results for confirming a lipid level in the liver, it can be seen with the naked eye that fatty liver was reduced in the experimental groups compared to the control.

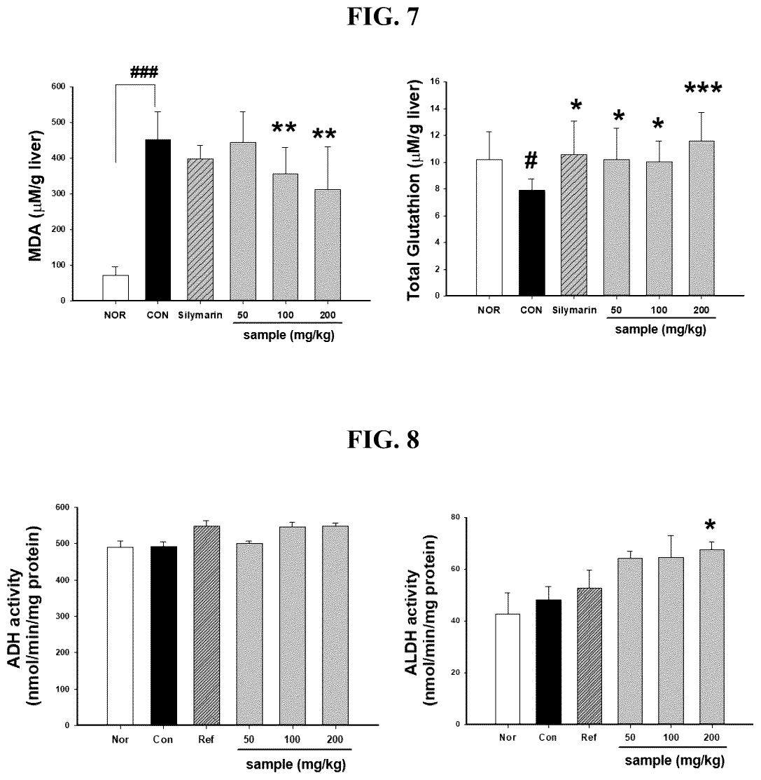

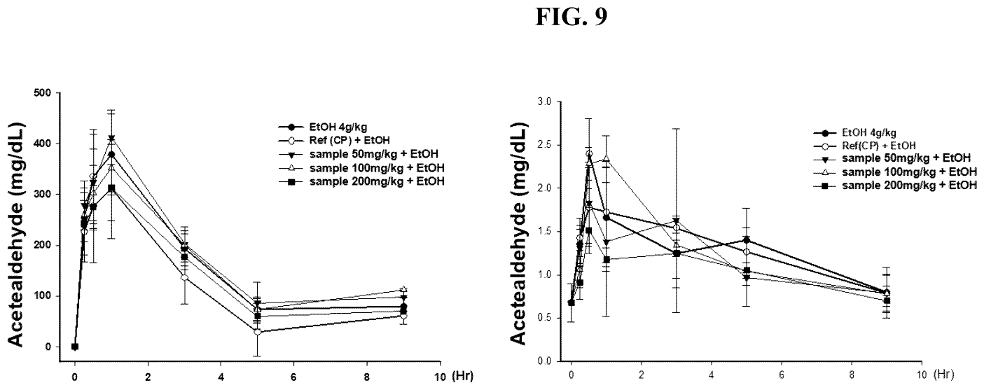

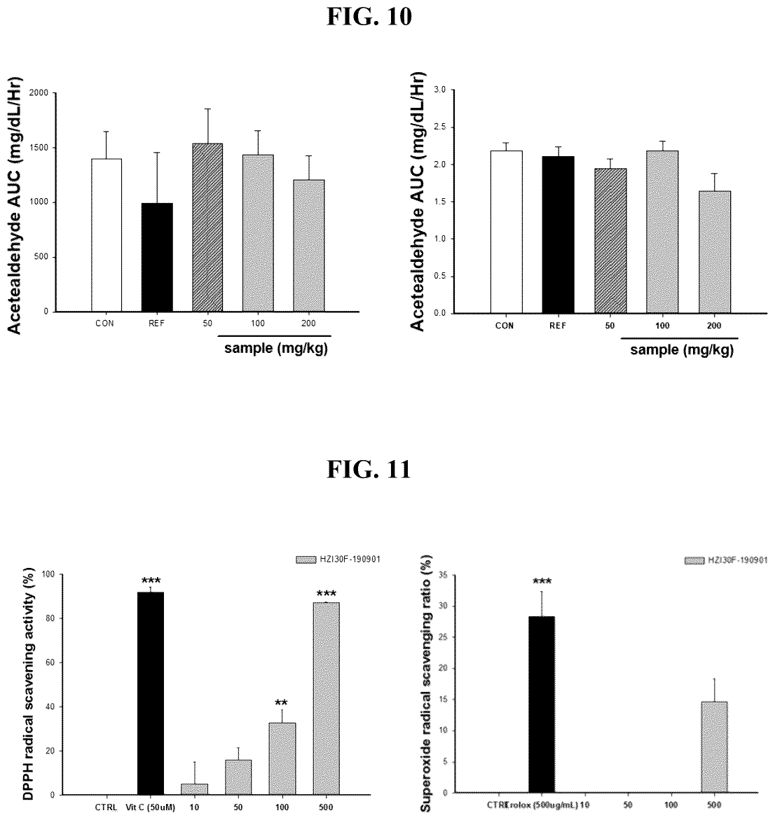

Meanwhile, the TG (triglyceride) and total cholesterol concentrations in liver tissue and serum (FIG. 6), the degrees of activation of lipid peroxide and non-enzymatic antioxidant systems in liver tissue (FIG. 7), the ADH and ALDH activities in liver tissue after alcohol administration (FIG. 8), and the changes in blood concentrations of alcohol and acetaldehyde (FIG. 9), and the Cₘₐₓ values of alcohol and acetaldehyde (FIG. 10) were measured.

Referring to the results shown in FIGS. 6 to 10, it was confirmed that the Zizania latifolia extract prepared in Preparation Example 1 had the effect of preventing or alleviating alcohol-induced fatty liver and liver damage in a concentration-dependent manner.

### Example 3: Evaluation of Antioxidant Ability of Zizania Latifolia Extract

The DPPH radical scavenging ability and superoxide radical scavenging ability were measured to evaluate the antioxidant ability of the Zizania latifolia extract prepared in Preparation Example 1 at each concentration, and the results are shown in FIG. 11. Referring to the results shown in FIG. 11, it can be seen that the Zizania latifolia extract exhibited antioxidant activity in a concentration-dependent manner, and particularly, the most superior antioxidant activity was expressed at 500 μM.

Referring to the various in vitro and in vivo experimental results as described above, it can be seen that the Zizania latifolia extract according to the present invention may exhibit a wide range of therapeutic or alleviating effects on non-alcoholic liver diseases and liver dysfunction, as well as alcohol-induced liver damage.

As described above, although the present invention has been described with reference to embodiments thereof, it should be understood by those of ordinary skill in the art that the present invention may be modified and changed in various ways by adding, changing, or deleting a component without departing from the spirit of the present invention defined in the appended claims, and such modifications and changes are also be included in the scope of the present invention.

What is claimed is:

1. A pharmaceutical composition for treating or ameliorating a liver disease and liver dysfunction, comprising a Zizania latifolia extract.

2. The pharmaceutical composition of claim 1, wherein the liver disease is at least one selected from fatty liver, liver fibrosis, liver cirrhosis, liver cancer, jaundice, and an inflammatory liver disease.

3. The pharmaceutical composition of claim 1, wherein the inflammatory liver disease is at least one selected from hepatitis, acute hepatitis, chronic hepatitis, alcoholic hepatitis, non-alcoholic hepatitis, subacute hepatitis, viral hepatitis, toxic liver disease, liver abscesses, granulomatous hepatitis, autoimmune hepatitis, and lupoid hepatitis.

4. The pharmaceutical composition of claim 1, wherein the liver dysfunction is caused by at least one selected from hangovers, alcohol-induced liver damage, toxic substance-induced liver damage, and hepatocyte damage.

5. The pharmaceutical composition of claim 1, wherein the Zizania latifolia extract comprises:
an enzyme-treated extract of Zizania latifolia Turcz.; and
a polar or non-polar solvent extract of the residue remaining after obtaining the enzyme-treated extract of Zizania latifolia Turcz.

6. The pharmaceutical composition of claim 5, wherein the enzyme-treated extract of Zizania latifolia Turcz. is obtained by treating Zizania latifolia Turcz. with at least one enzyme selected from a pectinase, a hemicellulase, an arabinanase, an arabanase, a cellulase, a beta-glucanase, and a xylanase.

7. The pharmaceutical composition of claim 1, wherein the Zizania latifolia extract comprises at least one selected from flavonoid glycosides and flavonoid aglycones.

8. The pharmaceutical composition of claim 1, wherein the Zizania latifolia extract comprises at least one selected from tricin, tricin-7-O-β-D-glucopyranose, salcolin A, sacolin B, sacolin C, salcolin D, tricin-4′-O-(threo-β-guaiacylglyceryl) ether 7-O-β-D-glucopyranose, tricin-4′-O-(erythro-β-guaiacylglyceryl) ether 7-O-β-D-glucopyranose, tricin-4′-O-(threo-β-guaiacylglyceryl) ether 7″-O-β-D-glucopyranose, and tricin-4′-O-(erythro-β-guaiacylglyceryl) ether 7″-O-β-D-glucopyranose.

9. The pharmaceutical composition of claim 1, further comprising at least one selected from a vitamin B complex, vitamin C, vitamin E, beta-carotene, Ca, Mg, Zn, lecithin, alanine, taurine, maltol, fructose, an oligosaccharide, Ganoderma lucidum, glutamate, chitosan, aspartic acid, Cordyceps militaris, a Hovenia dulcis extract, an Alnus borealis extract, and a milk thistle extract.

10. The pharmaceutical composition of claim 1, wherein the Zizania latifolia extract comprises 0.1% by weight to 90% by weight of tricin.

11. A food composition for ameliorating or preventing a liver disease and liver dysfunction, comprising a Zizania latifolia extract.

12. The food composition of claim 11, wherein the food composition is a health functional food.
